(19) United States
(12) Patent Application Publication
(10) Pub. No.: US 2005/0145256 A1
(43) Pub. Date: Jul. 7, 2005

(54) Orthopedic walker having a soft boot with a deformable insert

(76) Inventors: Mark E. Howard, San Diego, CA (US); Sean B. Cahill, Temecula, CA (US)

Correspondence Address: Rodney F. Brown, 3365 Baltimore Street, San Diego, CA, 92117, US

(21) Appl. No.: 10/745,900

(22) Filed: Dec. 24, 2003

(51) Int. Cl. A61F 5/37

(52) U.S. Cl. 128/882

(57) ABSTRACT

An orthopedic walker has a rigid external shell, a pliant internal boot positioned in the external shell, and a deformable insert positioned within the internal boot. The internal boot is sized to enclose a lower extremity of a user. The deformable insert has a heel segment including a fully-encircled heel opening. The deformable insert freely rests within the internal boot and is sized and positioned to receive the heel of the lower extremity within the heel opening. However, the deformable insert is sufficiently undersized to maintain a continuous exposed surface extending between a top surface of the foot and a front surface of the ankle.

### TECHNICAL FIELD

The present invention relates generally to orthopedic devices, and more particularly to orthopedic walkers for supporting and/or immobilizing the lower extremity of a user during activity.

### BACKGROUND OF THE INVENTION

Orthopedic walkers are designed to be worn on the lower extremity of a patient afflicted with a lower leg, ankle, or foot injury, such as a fracture, soft tissue injury, or the like. The walker immobilizes and supports the injured lower extremity during low-impact, weight-bearing activities such as walking and standing. The walker is often an effective substitute for a conventional plaster or fiberglass cast with numerous advantages. Among its advantages, walkers are prefabricated and adaptable to different legs, are more comfortable to wear, are more easily cleaned, and can be temporarily removed by the patient for bathing, sleeping, range of motion exercise, or other non-weight-bearing activities.

Many orthopedic walkers are known in the prior art as evidenced by the following representative U.S. Pat. No. 5,078,128 to Grim et al., 4,771,768 to Crispin, 4,057,056 to Payton, and 3,955,565 to Johnson, Jr. Nevertheless, the present invention recognizes a need for an orthopedic walker which is an improvement over those of the prior art. Accordingly, it is an object of the present invention to provide an improved orthopedic walker. More particularly, it is an object of the present invention to provide an orthopedic walker having an improved fit to the lower extremity of the patient. These objects and others are accomplished in accordance with the invention described hereafter.

### SUMMARY OF THE INVENTION

In accordance with a preferred embodiment, the present invention is an orthopedic walker comprising a rigid external shell, a pliant internal boot positioned in the external shell, and a deformable insert positioned within the internal boot. The internal boot has a foot portion, a leg portion and a heel portion, which are sized to enclose a lower extremity of a user and, in particular, to enclose the foot, lower leg including the ankle, and heel of the lower extremity. The deformable insert has a foot segment, an ankle segment, and a heel segment, which includes a fully-encircled heel opening. The deformable insert freely rests within the internal boot and is distributed about the heel portion in direct engagement with the internal boot.

The deformable insert is sized and positioned in the internal boot to directly engage the foot and ankle proximal to the heel. The deformable insert is further sized and positioned to receive the heel within the fully-encircled heel opening. However, the deformable insert is sufficiently undersized to maintain a continuous exposed surface of the lower extension which extends between a top surface of the foot and a front surface of the ankle when the deformable insert engages the heel, foot and ankle.

In accordance with more specific embodiments, the deformable insert is either a fluid-containing bladder, an evacuatable bead-containing bladder, or an inflatable bladder. In the case of an evacuatable or inflatable bladder, the deformable insert has a valve stem, which houses a fluid valve and extends from the deformable insert. A valve orifice is formed through the internal boot, which is sized and positioned for receiving the valve stem through the internal boot to externally access the valve stem.

In accordance with an alternate embodiment, the orthopedic walker further comprises an outer sleeve having the deformable insert received therein. The outer sleeve has an outer foot segment, an outer ankle segment and an outer heel segment. The outer sleeve freely rests within the internal boot distributed about the heel portion and in direct engagement with the internal boot. The outer sleeve is sized and positioned in the internal boot to directly engage the foot and ankle proximal to the heel. The outer heel segment also preferably includes a fully-encircled outer heel opening to receive the heel. The outer sleeve is preferably sufficiently undersized to maintain a continuous exposed surface of the lower extremity, which extends between a top surface of the foot and a front of the ankle when the outer sleeve engages the heel, foot and ankle.

In accordance with yet another embodiment, the present invention is a method for fitting an orthopedic walker to a lower extremity. A pliant internal boot is provided having a foot portion, a leg portion, and a heel portion, which enclose a boot interior. The internal boot is initially opened to expose the boot interior. A deformable insert is positioned within the boot interior and distributed about the heel portion of the internal boot. The deformable insert has a foot segment, an ankle segment, and a heel segment, which includes a fully-encircled heel opening.

The lower extremity is displaced into the boot interior causing the foot and ankle to engage the foot and ankle segments, respectively, of the deformable insert. When the heel is received into the heel opening, the internal boot is closed around the lower extremity and the deformable insert. The foot segment wraps around an underside of the foot and the ankle segment wraps around a backside of the ankle. The deformable insert is sufficiently undersized to maintain a continuous exposed surface of the lower extremity which extends from a top surface of the foot to an anterior surface of the ankle.

A rigid external shell is further provided having a plurality of retention straps. The internal boot containing the lower extremity and deformable insert is placed within the external shell. The retention straps are tensioned to secure the internal boot within the external shell.

In accordance with a more specific embodiment, the deformable insert is an inflatable bladderand the fitting method further comprises adding fluid to orwithdrawing fluid from the inflatable bladder after positioning the internal boot in the external shell or, alternatively, after tensioning the retention straps.

The present invention will be further understood from the drawings and the following detailed description.

### BRIEF DESCRIPTION OF THE DRAWINGS

FIG. 1 is a side perspective view of an orthopedic walker of the present invention.

FIG. 2 is a front perspective view of the orthopedic walker of FIG. 1.

FIG. 3 is a top perspective view of an external shell employed in the orthopedic walker of FIG. 1.

FIG. 4 is a top perspective view of an insole employed in the orthopedic walker of FIG. 1.

FIG. 5 is a front perspective view of a deformable insert resting in an internal boot of the orthopedic walker of FIG. 1.

FIG. 6 is a side perspective view of the deformable insert of FIG. 5.

FIG. 7 is a perspective view of the deformable insert conformed to the foot and ankle of a lower extremity within the internal boot of FIG. 5.

FIG. 8 is a side perspective view of an alternate embodiment of the present invention wherein the deformable insert of FIG. 5 is fitted within an outer sleeve.

### DESCRIPTION OF PREFERRED EMBODIMENTS

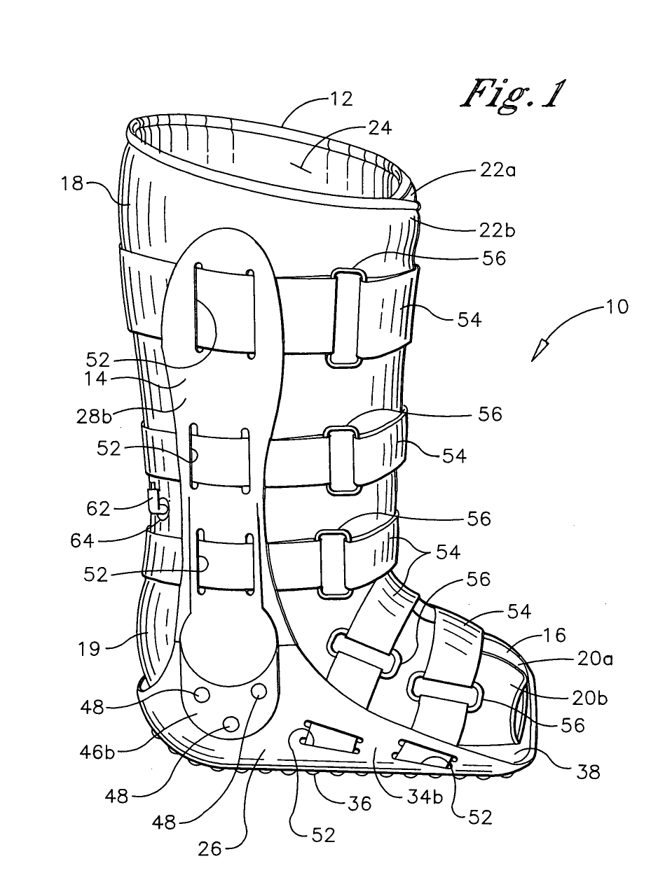

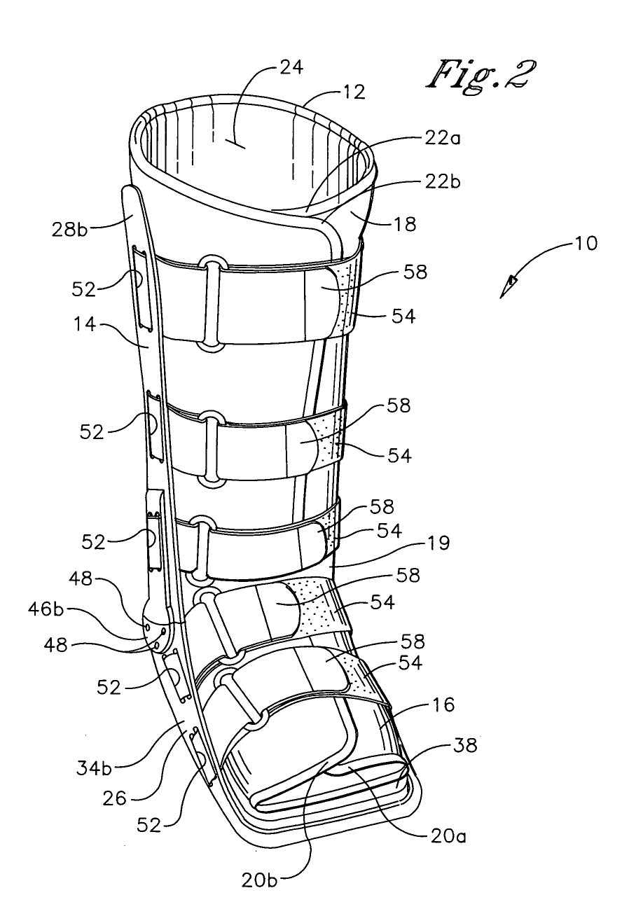

Referring initially to FIGS. 1 and 2, an embodiment of the orthopedic walker of the present invention is shown and generally designated 10. The orthopedic walker 10 comprises an internal boot 12 and an external shell 14. The internal boot 12 is generally sized and configured to be worn on a lower extremity of a user (not shown) in the manner of conventional knee-high footwear. As such, the internal boot 12 has a foot portion 16 fitting over the foot of the lower extremity, a leg portion 18 fitting over the lower leg (including the ankle) of the lower extremity, and a heel portion 19 fitting over the heel of the lower extremity. In particular, the foot portion 16 extends forward from the ankle over the toes, the leg portion 18 extends upward from the ankle to just below the knee, and the heel portion 19 is at the junction of the foot and leg portions 16, 18, which corresponds in position to the heel of the lower extremity.

The internal boot 12 is fabricated from a soft pliant material, which insulates the enclosed lower extremity from direct contact with rigid objects external to the internal boot 12, thereby both cushioning and protecting the lower extremity. A preferred material for the internal boot 12 is a sheet of cloth-foam-cloth laminate with the cloth forming the exposed faces of the laminate. The internal boot 12 is constructed by cutting and sewing the laminate into the boot configuration. The internal boot 12 is preferably free of any supplemental support structures, such as stays, stiffeners, splints, fluid-filled bladders, or the like, which are integral with or attached to the internal boot 12. Thus, the internal boot 12 preferably lacks any retention structures, such as stitching, pockets, straps, fasteners, or the like, which are adapted to retain any supplemental support structures.

The anterior of the internal boot 12 is provided with first and second selectively engagable lower flaps 20a, 20b and first and second selectively-engagable upper flaps 22a, 22b. The lower flaps 20a, 20b are retained in engagement with one another by releasable fasteners (not shown), such as conventional hook and loop fasteners commercially available under the trade name VELCRO. The upper flaps 22a, 22b are similarly retained in engagement with one another by releasable fasteners. In a preferred embodiment, the cloth forming the exterior of the internal boot 12 constitutes the hook material of the hook and loop fasteners.

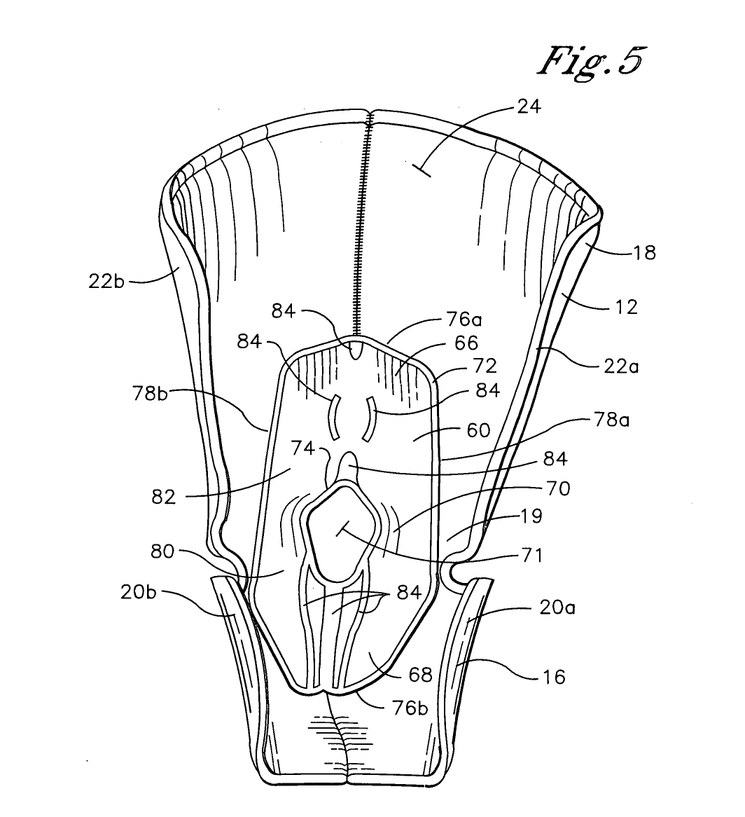

The lower flaps 20a, 20b are disengaged from engagement with one another by releasing the fasteners and pulling the first lower flap 20a apart from second lower flap 20b. The upper flaps 22a, 22b are similarly selectively disengaged from engagement with one another by releasing the fasteners and pulling the first upper flap 22a apart from second upper flap 22b. Disengagement of the lower and upper flaps 20a, 20b, 22a, 22b opens up the anterior of the internal boot 12 (as shown in FIG. 5) to receive the lower extremity into the interior 24 of the internal boot 12. Once the internal boot 12 is fitted around the lower extremity, the lower and upper flaps 20a, 20b, 22a, 22b may be re-engaged to retain the internal boot 12 on the lower extremity. In particular, the lower flaps 20a, 20b are re-engaged by sequentially closing the first lower flap 20a over the foot, closing the second lower flap 20b over the first lower flap 20a, and coupling the fasteners. The upper flaps 22a, 22b are similarly re-engaged by sequentially closing the first upperflap 22a overthe lower leg, closing the second upper flap 22b over the first upper flap 22a, and coupling the fasteners.

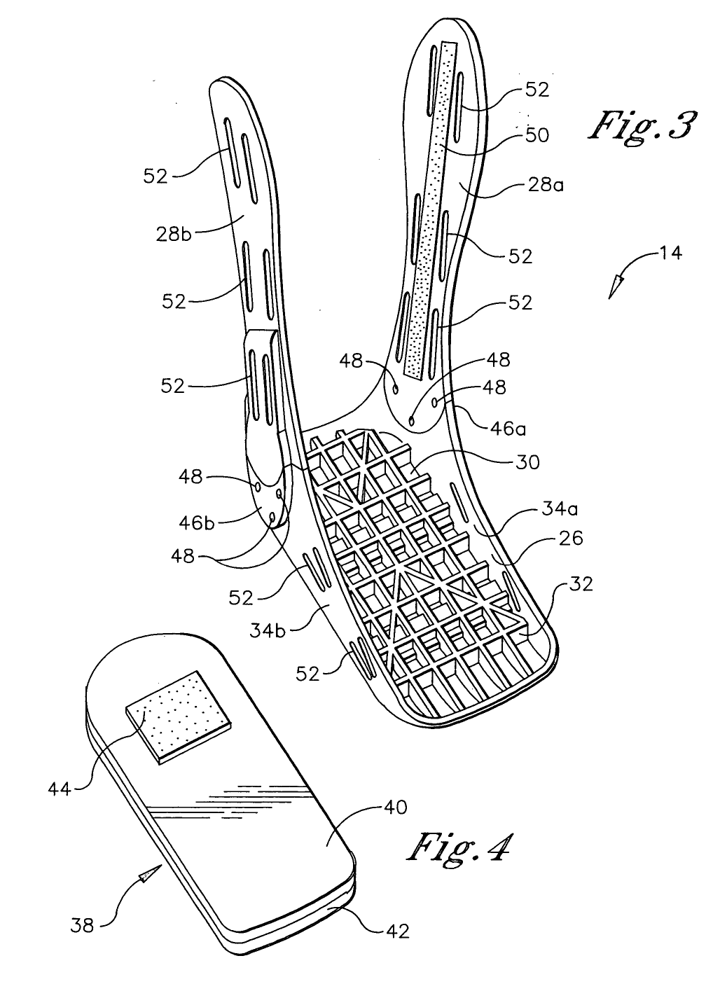

The external shell 14 is sized and configured in correspondence with the internal boot 12 so that the internal boot 12 fits within the external shell 14 while the internal boot 12 is worn on the lower extremity. Referring additionally to FIG. 3, the external shell 14 comprises a base assembly 26 and first and second uprights 28a, 28b. The elements 26, 28a, 28b of the external shell 14 are formed from a hard rigid material, such as metal, molded plastic, or fiberglass, which is preferably further characterized as being high-strength and lightweight. The terms “pliant” and “rigid” as used herein are relative terms. A rigid material, such as the material of the external shell 14, has sufficient stiffness to substantially support and/or immobilize the lower extremity so that a user having the affected lower extremity can engage in low-impact activities such as walking or standing without substantially stressing or otherwise further negatively affecting the lower extremity. Conversely, a pliant material, such as the material of the internal boot 12, by itself does not sufficiently support and/or immobilize the lower extremity to provide the affected lower extremity with any significant independent rehabilitative benefit.

The base assembly 26 is a unitary integral structure having a sole plate 30, a rib network 32, and first and second sidewalls 34a, 34b. The sole plate 30 is a continuous piece extending along the bottom of the base assembly 26 and generally dimensioned in correspondence with the length and width of the foot. As such, the sole plate 30 extends essentially the entire length of the foot portion 16 of the internal boot 12 when the internal boot 12 is worn on the lower extremity and fitted within the external shell 14. Tread 36 is provided on the bottom side of the sole plate 30 and the sole plate 30 is bowed slightly upward from its center to facilitate the function of the sole plate 30 as a walking platform for the foot against a walking surface.

The rib network 32 extends from the top side of the sole plate 30 to enhance the structural rigidity of the sole plate 30. The rib network 32 prevents flexion of the sole plate 30 when functioning as a walking platform. Referring additionally to FIG. 4, an insole 38 is preferably positioned atop the rib network 32 opposite the sole plate 30 to supplement the cushioning of the internal boot 12 along the sole of the foot when the internal boot 12 is worn on the lower extremity and fitted within the external shell 14. The insole 38 is preferably fabricated from a foam-cardboard laminate with a foam layer 40 mounted atop a cardboard layer 42. A releasable fastener 44 is affixed to the insole 38 to selectively retain the foot portion 16 of the internal boot 12 in engagement with the insole 38 and correspondingly in connection with the external shell 14. The releasable fastener 44 is a patch of loop material, while the exterior cloth of the internal boot 12 constitutes the hook material of a resulting hook and loop fastener.

The first and second sidewalls 34a, 34b extend upward from the opposing lateral and medial sides of the sole plate 30, respectively, adjacent to the ankle when the internal boot 12 is worn on the lower extremity and fitted within the external shell 14. The first sidewall 34a has a first mount 46a formed thereon, to which the first upright 28a is fixably mounted by fixable fasteners 48, such as rivets. The second sidewall 34b similarly has a second mount 46b formed thereon, to which the second upright 28b is fixably mounted by fixable fasteners 48. As such, the first and second mounts 46a, 46b provide substantially immobile joints between the first upright and sidewall 28a, 34a and the second upright and sidewall 28b, 34b, respectively. The first and second uprights 28a, 28b extend upward from the first and second mounts 38a, 38b, respectively, essentially the entire length of the leg portion 18 of the internal boot 12 when the internal boot 12 is worn on the lower extremity and fitted within the external shell 14. Releasable fasteners 50 are affixed to the inside faces of the first and second uprights 28a, 28b to selectively retain the leg portion 18 of the internal boot 12 in engagement with the external shell 14. Each releasable fastener 50 is preferably a patch of loop material, while the exterior cloth of the internal boot 12 constitutes the hook material of a resulting hook and loop fastener.

A plurality of strap slots 52 are provided in the uprights 28a, 28b and the sidewalls 34a, 34b, through which a plurality of retention straps 54 are threaded. Each retention strap 54 has a strap loop 56 attached to one end and a releasable fastener 58 attached to the opposite end. The releasable fastener 58 is preferably a patch of loop material, while the exterior of the retention strap 54 is covered with a hook material resulting in a hook and loop fastener. The ends of the retention strap 54 are releasably attached to each other by threading the releasable fastener 58 through the strap loop 56 and folding the releasable fastener 58 back onto the exterior of the retention strap 54. When it is desired to fit the internal boot 12 within the external shell 14, while the internal boot 12 is worn on the lower extremity, the fasteners 58 of the retention straps 54 are all released and threaded out of the strap loops 56 to open up the anterior of the external shell 14. The internal boot 12, while worn on the lower extremity, is then placed in the external shell 14 and all of the retention straps 54 are reattached in the above-recited manner. The retention straps 54 securely retain the internal boot 12 within the external shell 14 to any desired degree of tightness by selectively adjusting the tension of the retention straps 54 in a manner readily apparent to the skilled artisan. As such, the external shell 14 constitutes a rigid support frame for the pliant internal boot 12.

Referring to FIG. 5, the orthopedic walker 10 further comprises a deformable insert 60, which is sized and configured to rest freely within the interior 24 of the internal boot 12, preferably neither attached nor otherwise connected to the internal boot 12 or external shell 14. In particular, the deformable insert 60 is distributed about the heel portion 19 within the interior 24 of the internal boot 12. The deformable insert 60 is preferably deformable with respect to the dimension of thickness, but not necessarily with respect to dimensions of length and width. In particular, the thickness of the deformable insert 60 can preferably be varied in response to applied pressure. More particularly, the localized thickness of the deformable insert 60 can be decreased significantly in response to application of a sufficient degree of localized pressure.

The deformable insert 60 contains a filler material (i.e., deformation medium), which preferably deforms by displacement rather than compression. In other words, the filler material is flowable, but relatively incompressible, when subjected to the range of pressures normally encountered in orthopedic applications. Accordingly, when the thickness of the deformable insert 60 is decreased at one location, the thickness of the deformable insert 60 increases at an adjoining location. In a preferred embodiment, the total volume of the deformable insert 60 remains essentially constant during normal user activity of the orthopedic walker. However, in a preferred embodiment, the total volume of the deformable insert 60 can be varied by adding filler material to or removing filler material from the deformable insert 60 during a user fitting procedure described below. A preferred filler material having the requisite properties is a fluid, wherein the term “fluid” is inclusive of gases and liquids and, unless stated otherwise, is further inclusive of flowing gels and flowing foams.

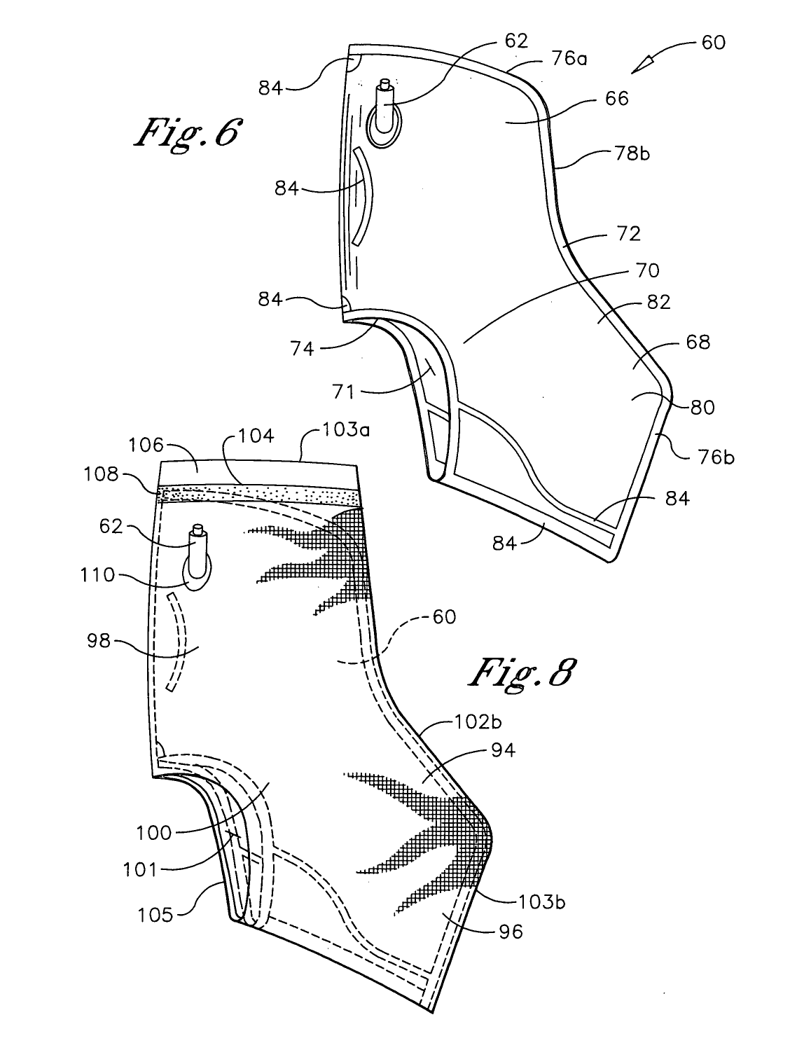

With added reference to FIG. 6, the deformable insert 60 of a preferred embodiment is an inflatable bladder, which has a fluid valve (not shown) housed within a valve stem 62. The fluid valve may be selectively opened to enable the addition or withdrawal of fluid filler material, typically air, into or out of the inflatable bladder 60 during the user fitting procedure. However, the fluid valve is preferably maintained closed to prevent the addition or withdrawal of fluid filler material during normal user activity. The valve stem 62 has a stovepipe configuration for clearance from the inflatable bladder 60. When the inflatable bladder 60 rests within the interior 24 of the internal boot 12, a valve orifice 64 (shown in FIG. 1) formed in the leg portion 18 of the internal boot 12 is aligned with and receives the valve stem 62 so that the fluid valve is freely accessible to the outside for the addition or withdrawal of fluid, even while the orthopedic walker 10 is being worn on the affected lower extremity.

The inflatable bladder 60 preferably has a configuration which enables correspondence with the configuration of the internal boot 12 and similarly with the configuration of a foot, ankle and heel of a lower extremity as described below. The inflatable bladder 60 comprises a foot segment 66 and an ankle segment 68, which are aligned at approximately 450 to each other. The inflatable bladder 60 further comprises a heel segment 70 at the intersection of the foot and ankle segments 66, 68, which includes a heel opening 71. As such, the foot segment 66 is positionable forward of the heel portion 19 of the internal boot 12, the ankle segment 68 is positionable above the heel portion 19 of the internal boot 12, and the heel segment 70 is positionable at the heel portion 19 of the internal boot 12.

The inflatable bladder 60 is preferably constructed from two identical sections of pliant, fluid-tight, smooth plastic sheeting, which are loosely positioned one atop the other with a substantial degree of slack and joined together at their edges by welding or the like to form a fluid-tight outer peripheral seam 72 and a fluid-tight inner peripheral seam 74. As such, the outer peripheral seam 72 tracks the four outer edges of the resulting inflatable bladder 60, i.e., the top and bottom outer edges 76a, 76b and the lateral and medial outer edges 78a, 78b. The inner peripheral seam 74 tracks the inner edge of the inflatable bladder 60 and correspondingly defines the outer edge of the heel opening 71. The heel opening 71 is fully encircled without substantial interruption by the inner peripheral seam 74.

The joined plastic sheeting forms the wall 80 of the inflatable bladder 60 and the exposed outer faces of the plastic sheeting become the external surface 82 of the inflatable bladder 60. Additional conformance seams 84 may be provided along the interior of the inflatable bladder 60 away from the edges. The conformance seams 84 facilitate bending of the inflatable bladder 60 to conform the inflatable bladder 60 to the contours of the foot and ankle in a manner described below when the inflatable bladder 60 contains a fluid.

Referring to FIG. 7, the inflatable bladder 60 is shown during normal user activity when the internal boot 12 is worn on the lower extremity 85. The inflatable bladder 60 is sized and configured to conform to the heel 86 and to the proximal surrounding areas of the foot 88 and ankle 90. In particular, the inflatable bladder 60 is sized and configured to engage only limited surfaces of the foot 88, ankle 90 and heel 86. With the inflatable bladder 60 resting in the interior 24 of the internal boot 12, the heel 86 is received into the heel opening 71 with the inner peripheral seam 74 engaging the heel 86. The remainder of the heel 86 is free from engagement with the inflatable bladder 60 and extends through the heel opening 71 where the heel 86 is exposed to direct engagement with the internal boot 12. When the internal boot 12 is closed over the foot 88, ankle 90 and heel 86, the foot segment 66 wraps around the underside of the foot 88 proximal to and forward of the heel 86, thereby engaging the lateral, medial, and bottom surfaces of the foot 88. The ankle segment 68 similarly wraps around the backside of the ankle 90 proximal to and above the heel 86, thereby engaging the lateral, medial, and posterior surfaces of the ankle 90.

The inflatable bladder 60 is sufficiently undersized so that the lateral and medial outer edges 78a, 78b do not converge onto one another when the heel 86 is received in the heel opening 71 and the foot and ankle segments 66, 68 wrap around the foot 88 and ankle 90, respectively. Consequently, a continuous exposed surface 92 of the foot 88 and ankle 90 remains free from engagement with the inflatable bladder 60 between the lateral and medial outer edges 78a, 78b thereof. The exposed surface 92 extends from the top surface of the foot 88 to the anterior surface of the ankle 90 and is exposed to direct engagement with the internal boot 12.

The primary function of the deformable insert 60 is to enhance the close fit of the internal boot 12 to the foot 88 and ankle 90, which is beneficial to the overall therapeutic or rehabilitative function of the orthopedic walker 10. The deformable insert 60 is preferably not intended by itself to provide any significant medical benefit to the affected lower extremity independent of the internal boot 12 and external shell 14.

The deformable insert 60 preferably lacks any active means, such as straps, fasteners, or the like, for attaching or otherwise connecting the deformable insert 60 to the internal boot 12 or external shell 14. The deformable insert 60 likewise preferably lacks any active means, such as straps, fasteners, or the like, for retaining the deformable insert 60 in engagement with the foot 88, ankle 90, or heel 86. The internal boot 12 is the primary active means for retaining the deformable insert 60 in engagement with the foot 88, ankle 90 and heel 86. When the deformable insert 60 is separate from the internal boot 12, the deformable insert 60 does not retain engagement with the foot 88, ankle 90 or heel 86, but simply disengages when the foot 88, ankle 90 and heel 86 are moved.

The fitted configuration of the deformable insert 60 enhances the ability of the orthopedic walker 10 to maintain the relative positioning of the deformable insert 60 and the foot 88, ankle 90 and heel 86 in the interior 24 of the internal boot 12 in the absence of any active connective means. Specifically, the inner peripheral seam 74 functions as a reference guide for precise positioning of the deformable insert 60 relative to the foot 88, ankle 90 and heel 86. Furthermore, once the heel opening 71 receives the heel 86, the inner peripheral seam 74 passively “grips” the heel 86, which limits undesirable movement of the deformable insert 60 relative to the lower extremity 85 when the lower extremity 85 and deformable insert 60 reside in the internal boot 12.

In practice, the user prepares the orthopedic walker 10 for normal user activity by performing the user fitting procedure. The user fitting procedure is initiated by opening up the anterior of the internal boot 12 by disengaging the flaps 20a, 20b, 22a, 22b and resting the deformable insert 60 in the interior 24 of the internal boot 12 at the heel as shown in FIG. 5. If the deformable insert 60 is an inflatable bladder, as described above, the inflatable bladder is first preferably deflated by withdrawing air from the inflatable bladder via the fluid valve until the inflation level of the inflatable bladder is substantially below a predicted optimum-fit inflation level. The lower extremity is displaced into the internal boot 12 until the heel engages the heel segment 70 of the deformable insert 60 and is received in the heel opening 71, the foot engages the foot segment 66 of the deformable insert 60, and the ankle engages the ankle segment 68 of the deformable insert 60. The anterior of the internal boot 12 is then closed over the lower extremity by re-engaging the flaps 20a, 20b, 22a, 22b.

The anterior of the external shell 14 is opened up by threading the retention straps 54 out of the strap loops 56. The internal boot 12, while worn on the lower extremity and containing the deformable insert 60, is then placed in the external shell 14. All of the retention straps 54 are reattached at a desired tension to provide a close fit between the external shell 14 and the internal boot 12 and between the internal boot 12 and the lower extremity. If the deformable insert 60 is an inflatable bladder, an optimal close fit between the internal boot 12 and the foot and ankle is effected after reattaching the retention straps 54 by preferably inflating air into the inflatable bladder via the fluid valve by means of a pump (not shown) until the inflation level of the inflatable bladder reaches an actual optimum-fit inflation level. It is noted that in some cases it may alternatively be necessary to deflate, rather than inflate, the inflatable bladder to achieve the actual optimum-fit inflation level. In any case, the wall of the inflatable bladder preferably exhibits substantial slack at the actual optimum-fit inflation level to permit deformation of the bladder wall and the enclosed fluid. The user fitting procedure is completed upon adjustment of the inflatable bladder inflation level (or reattachment of the retention straps if the deformable insert is not inflatable).

The user may perform normal user activity while wearing the adjustably fitted orthopedic walker 10 after the user fitting procedure is completed. If it is desired to readjust the fit of the orthopedic walker 10 once normal user activity has commenced, the user simply interrupts the normal user activity and adjusts the inflation level of the inflatable bladder to a new desired inflation level (or reattaches the retention straps at a new desired tension if the deformable insert is not inflatable) and resumes normal user activity thereafter.

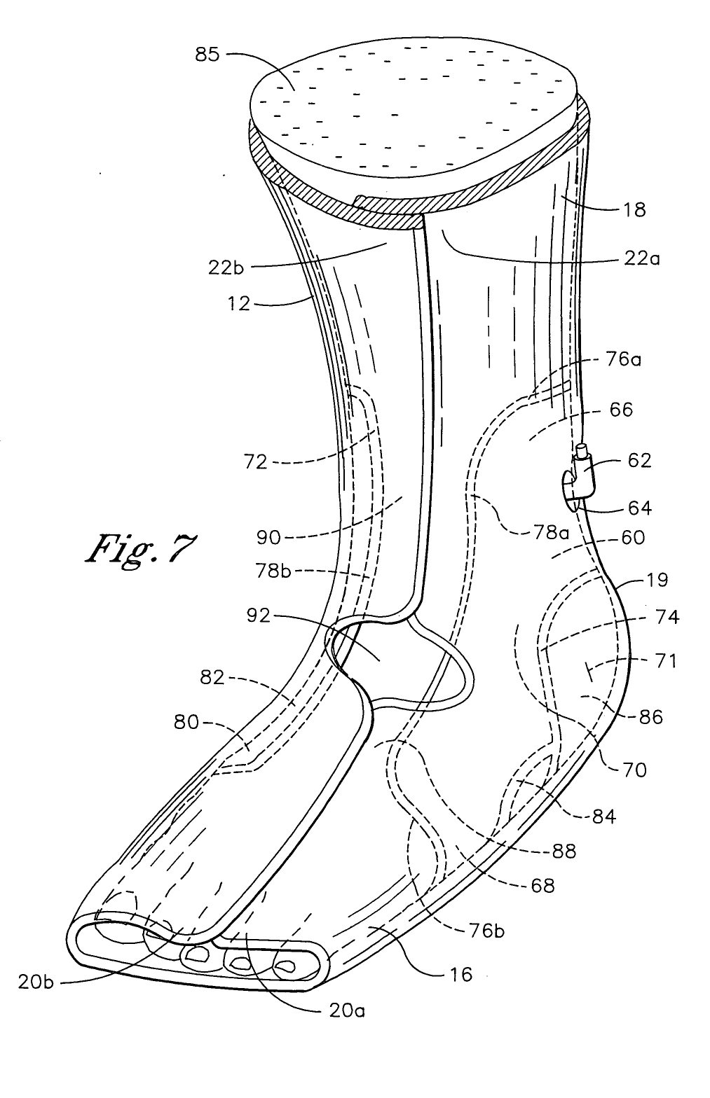

Although the lower extremity 85 may directly engage the external surface 82 of the deformable insert 60, an alternate embodiment of the-present invention is provided, which insulates the lower extremity 85 from the external surface 82 of the deformable insert 60. The alternate embodiment is described with reference to FIG. 8, wherein elements corresponding to those of FIGS. 1-7 are identified by the same reference characters. The embodiment of FIG. 8 is identical in structure and practice to the embodiment of FIGS. 1-7 except that an outer sleeve 94 is additionally provided, which encloses the deformable insert 60. The outer sleeve 94 is fabricated from a soft pliable absorbent cloth and is sized and configured in correspondence with the size and shape of the deformable insert 60. As such, the outer sleeve 94 has an outer foot segment 96, an outer ankle segment 98 and an outer heel segment 100 including an outer heel opening 101, all of which align with the corresponding elements of the deformable insert 60 when the deformable insert 60 is received within the outer sleeve 94.

The outer sleeve 94 is preferably constructed as a pouch with three essentially permanently enclosed outer peripheral sides. In particular, the outer sleeve 94 has an enclosed medial side 102b, an enclosed lateral side (not shown) opposite the medial side 102b, and an enclosed bottom side 103b. The outer sleeve 94 also has a top side 103a, which has a top opening 104 for receiving the deformable insert 60 into the outer sleeve 94 or for removing the deformable insert 60 from the outer sleeve 94. The outer heel opening 101 has inner peripheral sides 105 which are preferably not enclosed so that the peripheral sides 105 do not impede the receipt or removal of the deformable insert 60 into or from the outer sleeve 94.

The outer sleeve 94 has a flap 106 which can be folded over the top opening 104 to selectively enclose the deformable insert 60 after the deformable insert 60 is received into the outer sleeve 94. A releasable fastener 108, such as a hook and loop fastener, is optionally provided at the top opening 104 to releasably retain the flap 106 against the outer sleeve 94 and maintain the top opening 104 closed, if desired. A valve orifice 110 is also provided through the outer sleeve 94 when the deformable insert 60 is an inflatable bladder having a valve stem 62.

The outer sleeve 94, like the deformable insert 60, preferably lacks any active means, such as straps, fasteners, or the like, for attaching or otherwise connecting the outer sleeve 94 to the internal boot 12 or external shell 14 and likewise preferably lacks any active means, such as straps, fasteners, or the like, for retaining the outer sleeve 94 in engagement with the foot 88, ankle 90 or heel 86. The fitted configuration of the outer sleeve 94, and in particular the heel opening 101, similarly enhances the ability of the orthopedic walker 10 to maintain the relative positioning of the deformable insert 60 and the foot 88, ankle 90 and heel 86 in the interior 24 of the internal boot 12 in the absence of any active connective means.

A preferred function of the outer sleeve 94 is to enhance user comfort. The outer sleeve 94 insulates the skin of the foot and ankle from the external surface 82 of the deformable insert 60 to avoid skin irritation. The outer sleeve 94 also absorbs any perspiration emanating from the foot or ankle when the user is wearing the orthopedic walker. The outer sleeve 94 by itself or in combination with the deformable insert 60 does not generally provide any significant independent rehabilitative benefit as a standalone support for the affected lower extremity.

One preferred embodiment of an external shell, i.e., a partially-enclosing external frame, has been described above for purposes of illustrating the present invention. However, it is understood that the present invention is not limited tojust the one specific embodiment of the external shell described herein, but is inclusive of other conventional rigid structures capable of supporting the internal boot. For example, U.S. Pat. No. 3,955,565 discloses an essentially fully-enclosing rigid casing, which may be adapted for utility herein as an alternate external shell of the present orthopedic walker. U.S. Pat. No. 3,955,565 is incorporated herein by reference.

Likewise, one preferred embodiment of a deformable insert, i.e., an inflatable bladder, has been described above for purposes of illustrating the present invention. However, it is understood that the present invention is not limited tojust the one specific embodiment of the deformable insert described herein, but is inclusive of other deformable structures capable of enhancing the fit of the internal boot to the foot and ankle. For example, alternate deformable inserts having utility herein include permanently sealed (i.e., non-inflatable) fluid pouches, such as gel packs, air pillows or the like. The outer wall of the permanently sealed fluid pouch preferably exhibits substantial slack at the permanent fill level to permit deformation of the outer wall and the enclosed fluid when pressure is applied similar to the manner of the inflatable bladders. Another alternate deformable insert of the present orthopedic walker is an evacuatable bladder containing solid, lightweight, compressible or incompressible beads as disclosed in U.S. Pat. No. 3,745,998 and 5,378,223, which may be adapted for utility herein. U.S. Pat. No. 3,745,998 and 5,378,223 are incorporated herein by reference.

While the forgoing preferred embodiments of the invention have been described and shown, it is understood that alternatives and modifications, such as those suggested and others, may be made thereto and fall within the scope of the invention.

1. An orthopedic walker comprising:
a rigid external shell;
a pliant internal boot positioned in said external shell, wherein said internal boot has a foot portion, a leg portion and a heel portion sized to enclose a foot, a lower leg including an ankle, and a heel of a lower extremity; and
a deformable insert having a foot segment, an ankle segment and a heel segment including a fully-encircled heel opening, wherein said deformable insert freely rests within said internal boot distributed about said heel portion and in direct engagement with said internal boot, wherein said deformable insert is sized and positioned in said internal boot to directly engage the foot and ankle proximal to the heel and to receive the heel within said fully-encircled heel opening, further wherein said deformable insert is sufficiently undersized to maintain a continuous exposed surface of the lower extremity extending between a top surface of the foot and a front of the ankle when said deformable insert engages the foot and ankle.

2. The orthopedic walker of claim 1 wherein said deformable insert is a fluid-containing bladder.

3. The orthopedic walker of claim 1 wherein said deformable insert is an evacuatable solid bead-containing bladder.

4. The orthopedic walker of claim 1 wherein said deformable insert is an inflatable bladder.

5. The orthopedic walker of claim 4 further comprising a valve stem extending from said inflatable bladder, wherein said valve stem houses a fluid valve.

6. The orthopedic walker of claim 5 further comprising a valve orifice formed through said internal boot, wherein said valve orifice is sized and positioned for receiving said valve stem through said internal boot to externally access said valve stem.

7. An orthopedic walker comprising:
a rigid external shell;
a pliant internal boot positioned in said external shell, wherein said internal boot has a foot portion, a leg portion and a heel portion sized to enclose a foot, a lower leg including an ankle, and a heel of a lower extremity;
a deformable insert having a foot segment, an ankle segment and a heel segment including a fully-encircled heel opening, wherein said deformable insert is positioned within said internal boot distributed about said heel portion, wherein said deformable insert is sized and positioned in said internal boot to receive the heel within said fully-encircled heel opening, and further wherein said deformable insert is sufficiently undersized to maintain a continuous exposed surface of the lower extremity extending between a top surface of the foot and a front of the ankle when said deformable insert engages the foot and ankle; and
an outer sleeve having said deformable insert received therein and having an outer foot segment, an outer ankle segment and an outer heel segment, wherein said outer sleeve freely rests within said internal boot distributed about said heel portion and in direct engagement with said internal boot, and further wherein said outer sleeve is sized and positioned in said internal boot to directly engage the foot and ankle proximal to the heel.

8. The orthopedic walker of claim 7 wherein said outer heel segment includes a fully-encircled outer heel opening to receive the heel.

9. The orthopedic walker of claim 7 wherein said outer sleeve is sufficiently undersized to maintain a continuous exposed surface of the lower extremity extending between a top surface of the foot and a front of the ankle when said outer sleeve engages the foot and ankle.

10. The orthopedic walker of claim 7 wherein said deformable insert is a fluid-containing bladder.

11. The orthopedic walker of claim 7 wherein said deformable insert is an evacuatable solid bead-containing bladder.

12. The orthopedic walker of claim 7 wherein said deformable insert is an inflatable bladder.

13. The orthopedic walker of claim 12 further comprising a valve stem extending from said inflatable bladder, wherein said valve stem houses a fluid valve.

14. The orthopedic walker of claim 13 further comprising a valve orifice formed through said internal boot, wherein said valve orifice is sized and positioned for receiving said valve stem through said internal boot to externally access said valve stem.

15. A method for fitting an orthopedic walker to a lower extremity comprising:
providing a pliant internal boot having a foot portion, a leg portion, and a heel portion enclosing a boot interior;
opening said internal boot to expose said boot interior;
positioning a deformable insert within said boot interior distributed about said heel portion, wherein said deformable insert has a foot segment, an ankle segment, and a heel segment including a fully-encircled heel opening;
displacing a lower extremity having a foot, a lower leg including an ankle, and a heel into said boot interior until said heel is received into said heel opening, said foot engages said foot segment, and said ankle engages said ankle segment;
closing said internal boot around said lowerextremity and said deformable insert to wrap said foot segment around an underside of said foot and said ankle segment around a backside of said ankle, wherein said deformable insert is sufficiently undersized to maintain a continuous exposed surface of said lower extremity extending from a top surface of said foot to an anterior surface of said ankle;
providing a rigid external shell having a plurality of retention straps;
positioning said internal boot containing said lower extremity and deformable insert within said external shell; and
tensioning said retention straps to secure said internal boot within said external shell.

16. The fitting method of claim 15 wherein said deformable insert is an inflatable bladder, the method further comprising adding fluid to or withdrawing fluid from said inflatable bladder after positioning said internal boot in said external shell.

17. The method of claim 15 wherein said deformable insert is an inflatable bladder, the method further comprising adding fluid to or withdrawing fluid from said inflatable bladder after tensioning said retention straps.
